(19) United States
(12) Patent Application Publication
(10) Pub. No.: US 2025/0195670 A1
(43) Pub. Date: Jun. 19, 2025

(54) METFORMIN NANOFORMULATIONS AND METHODS OF USE THEREOF

(71) Applicant: Board of Regents of the University of Nebraska, Lincoln, NE (US)

(72) Inventors: Chi Zhang, Omaha, NE (US); Tatiana Bronich, Brighton, MA (US); Fei Wang, Omaha, NE (US); Svetlana Romanova, Omaha, NE (US)

(21) Appl. No.: 18/846,364

(22) PCT Filed: Mar. 22, 2023
(86) PCT No.: PCT/US23/15870

§ 371 Date: Sep. 12, 2024

Related U.S. Application Data
(60) Provisional application No. 63/322,270, filed on Mar. 22, 2022.

(51) Int. Cl. A61K 47/59; A61K 47/60; A61P 35/00

(52) U.S. Cl. CPC A61K 47/595; A61K 47/60; A61P 35/00

(57) ABSTRACT

The present invention provides metformin nanoparticles and methods of use thereof.

This application claims priority under 35 U.S.C. § 119(e) to U.S. Provisional Patent Application No. 63/322,270, filed Mar. 22, 2022. The foregoing application is incorporated by reference herein.

### FIELD OF THE INVENTION

The present invention relates generally to the delivery of therapeutics. More specifically, the present invention relates to compositions and methods for the delivery of metformin to a patient.

### BACKGROUND OF THE INVENTION

Metformin is a commonly prescribed drug for the treatment of diabetes which reduces the liver's production of glucose and modulates a number of metabolic pathways. Metformin has been identified as a potential therapeutic candidate for the treatment of cancer due to its effects on both AMP-activated protein kinase (AMPK) dependent and independent mechanisms, its inhibitory effects on mitochondrial respiration, and the inhibition of mitochondrial glycerophosphate dehydrogenases. Metformin is also known to effect both monocyte differentiation into macrophages, as well as the secretion of proinflammatory cytokines. Metformin may be useful for modulating aberrant macrophage activity associated with a number of immunological disorders and diseases. However, one issue with the use of free metformin is that it does not accumulate in tumors and other tissues of interest at physiologically relevant concentrations when given at tolerated doses. Improved means of administering metformin are needed.

### SUMMARY OF THE INVENTION

In accordance with the instant invention, nanoparticles comprising a dendrimer covalently linked or attached to metformin are provided. In certain embodiments, the dendrimer is a poly(amidoamine) (PAMAM) dendrimer. In certain embodiments, the PAMAM dendrimer is a generation 3 PAMAM dendrimer, a generation 4 PAMAM dendrimer, or a generation 5 dendrimer. In certain embodiments, the PAMAM dendrimer is a generation 4 PAMAM dendrimer. In certain embodiments, the PAMAM dendrimer is conjugated to metformin via a linker. In certain embodiments, the linker comprises polyethylene glycol and/or a carbamate bond. In certain embodiments, the nanoparticle is P4-MET. In certain embodiments, the nanoparticle is PAMAM-PEG-MET. Compositions comprising at least one nanoparticle of the instant invention and at least one pharmaceutically acceptable carrier are also provided.

In accordance with another aspect of the instant invention, methods of treating, inhibiting, and/or preventing a disease or disorder in a subject in need thereof are provided. In certain embodiments, the method comprises administering to the subject at least one nanoparticle of the instant invention or a composition comprising the same. In certain embodiments, the disease is cancer. In certain embodiments, the cancer is a brain cancer such as a glioblastoma. In certain embodiments, the disease is neurodegenerative disease such as dementia including but not limited to Alzheimer's disease, traumatic brain injury, radiation- and/or chemotherapy-induced neurocognitive deficits. In certain embodiments, the method further comprises administering at least one additional therapy such as radiation therapy, chemotherapy, immunotherapy, and/or targeted therapy. In certain embodiments, the method further comprises administering radiation therapy.

### BRIEF DESCRIPTIONS OF THE DRAWING

FIG. 1 provides a graph of metformin release from P4-MET dendrimers over time (hours) at the indicated pH.

FIGS. 2A-2D provide graphs of U87MG growth at the indicated concentrations of metformin (FIG. 2A), PAMAM-OH (FIG. 2B), PAMAM-OH and metformin (FIG. 2C), and P4-MET (FIG. 2D).

FIGS. 3A-3D provide graphs of Ln229 growth at the indicated concentrations of metformin (FIG. 3A), PAMAM-OH (FIG. 3B), PAMAM-OH and metformin (FIG. 3C), and P4-MET (FIG. 3D).

FIGS. 4A-4D provide graphs of MGPP3 growth at the indicated concentrations of metformin (FIG. 4A), PAMAM-OH (FIG. 4B), PAMAM-OH and metformin (FIG. 4C), and P4-MET (FIG. 4D).

FIGS. 5A-5D provide graphs of bone marrow-derived macrophages (BMDM) growth at the indicated concentrations of metformin (FIG. 5A), PAMAM-OH (FIG. 5B), PAMAM-OH and metformin (FIG. 5C), and P4-MET (FIG. 5D).

FIG. 6A provides a graph of the survival of glioblastoma tumor mice treated with vehicle (Ctrl) or treated with metformin (Met) or P4-MET. ns=not significant. *p<0.05. FIG. 6B provides a graph of the body weight of glioblastoma tumor mice at the indicated times after tumor inoculation when untreated (Ctrl) or treated with metformin (Met) or P4-MET. FIG. 6C provides a graph of the survival of glioblastoma tumor mice treated with vehicle (Ctrl) or treated with metformin (Met) or P4-MET, optionally with brain radiation therapy (RT). Asterisks indicate degree of significance.

FIG. 7A provides the tissue (brain)/background ratio (TBR) of P4-MET-IR800 after one injection. P4-MET-IR800 or free dye (IR800) was injected into either control mice (No tumor) or glioblastoma tumor mice. FIG. 7B provides the tissue (brain)/background ratio (TBR) of P4-MET-IR800 in combination with brain radiation therapy (RT). P4-MET-IR800 or free dye (IR800) was injected into either control mice (No tumor) or glioblastoma tumor mice. Brain RT was administered at 24 hours after the initial injection of P4-MET-IR800 along with a second injection of P4-MET-IR800.

FIG. 8 provides the structure of PAMAM-OH G4.

### DETAILED DESCRIPTION OF THE INVENTION

The present invention describes nanoformulations of metformin that allow for rapid distribution of metformin into tissues of interest, increased cellular uptake, enhanced penetration across the blood-brain barrier, enhanced anti-tumor activity, and improved modulation of macrophage activity compared to free metformin. The compositions of the present invention can be used for the treatment of a variety of diseases such as cancer, diabetes and immunological disorders and diseases.

In accordance with the instant invention, nanoparticles (may also be referred to as a nanoformulations) comprising metformin are provided. In certain embodiments, the nanoparticles comprise a poly(amidoamine) (PAMAM) dendrimer conjugated and/or linked to metformin. Metformin may be conjugated and/or linked to the PAMAM dendrimer (e.g., at a terminal group via a functional group) either directly or via a linker. Dendrimers include molecular architectures with an interior core, layers/generations of repeating units which are attached to and extend from the interior core, and an exterior surface of terminal groups (e.g., attached to the outermost layer/generation). PAMAM dendrimers may comprise different cores and/or terminal groups, with the amidoamines.

In certain embodiments, the nanoparticles of the instant invention have a diameter (e.g., z-average diameter) or a longest dimension of less than about 500 nm, particularly less than about 100 nm, less than about 50 nm, or less than about 25 nm. In certain embodiments, the nanoparticles of the instant invention have a diameter of about 0.1 nm to about 50 nm, about 1 nm to about 25 nm, about 1 nm to about 20 nm, or about 1 nm to about 15 μm.

PAMAM dendrimers and methods of synthesis are well known in the art (e.g., de Araujo, et al., Molecules (2018) 23(11):2849; Maiti, et al., Macromolecules (2004) 37:6236-6254; Abbasi, et al., Nanoscale Res. Lett. (2014) 9:247; Santos, et al., Molecules (2016) 21:686; each incorporated by reference herein). For example, a layer/generation of the PAMAM dendrimer may be added by reacting an amino-terminated core or layer with methyl acrylate resulting in an ester-terminated outer layer, and reacting with ethylene diamine to obtain a new amino-terminated surface. The PAMAM dendrimers of the instant invention may be of any size/generation. In certain embodiments, the PAMAM dendrimer is from generation 1 to generation 10, particularly from generation 1 to generation 6, from generation 1 to generation 5, generation 2 to generation 6, generation 2 to generation 5, generation 3 to generation 6, or generation 3 to generation 5. In certain embodiments, the PAMAM dendrimer is generation 3, 4, or 5; generation 3 or 4; or generation 4 or 5. In certain embodiments, the PAMAM dendrimer is generation 4.

The PAMAM dendrimers of the instant invention may comprise one or more different terminal groups (surface group). In certain embodiments, the PAMAM dendrimer comprises one type of terminal group. Examples of PAMAM dendrimer terminal groups include, without limitation: amidoethanol terminal groups, amidoethylethanolamine terminal groups, amino terminal groups, hydroxyl terminal groups, hexylamide terminal groups, mixed (bi-functional) terminal groups, sodium carboxylate terminal groups, succinamic acid terminal groups, trimethoxysilyl terminal groups, tris(hydromethyl)amidomethane terminal groups, and 3-carbomethoxypyrrolidinone terminal groups. In certain embodiments, the terminal group comprises a hydroxyl group. In certain embodiments, the terminal group comprises an —NH₂ group.

Generally, the number of terminal groups is determined by the generation number of the PAMAM dendrimer. In certain embodiments, the PAMAM nanoparticles comprise between 1 and 5,000 termini groups. In certain embodiments, the PAMAM nanoparticles comprise between 1 and 1,000 termini groups. In certain embodiments, the PAMAM nanoparticles comprise between 1 and 100 termini groups.

The PAMAM dendrimer core comprises primary amines, particularly a linear chain molecules comprising primary amines. Examples of PAMAM core types include, without limitation: ammonia, ethylenediamine (2-carbon core), 1,4-diaminobutane (4-carbon core), 1,6-diaminohexane (6-carbone core), 1,12-diaminododecane (12-carbon core), and cystamine core (cleavable core). In certain embodiments, the PAMAM core is ethylenediamine.

As stated hereinabove, the PAMAM dendrimer is conjugated and/or linked to metformin, either directly or via a linker. In certain embodiments, metformin is conjugated and/or linked to the surface of the PAMAM dendrimer. In certain embodiments, metformin is conjugated and/or linked via a functional group (e.g., —OH or —NH₂) of the terminal group of the PAMAM dendrimer. In certain embodiments, the PAMAM dendrimer hydroxyl or amino surface groups are activated with 1,1′-carbonyldiimidazole to conjugate and/or link metformin. In certain embodiments, the PAMAM dendrimer is conjugated and/or linked to metformin via a crosslinker. In certain embodiments, the linker comprises at least one carbamate bond (—O(CO)NH—). In certain embodiments, the conjugation and/or linkage of metformin to the PAMAM dendrimer results in the formation of at least one carbamate bond (—O(CO)NH—).

In certain embodiments, the linker comprises polyethylene glycol (PEG). The PEG used may have a variety of ethylene glycol units. In certain embodiments, the PEG has between 1 and 100 ethylene glycol units. In certain embodiments, the PEG has between 1 and 10 ethylene glycol units.

The PAMAM dendrimers of the instant invention are conjugated and/or linked to one or more molecules of metformin. Metformin may be conjugated and/or linked to one, all, or any number of the terminal groups or surface functional groups of the PAMAM dendrimer. For example, with a generation 4 (G4) PAMAM dendrimer, there may be 64 terminal groups for attachment of metformin. Metformin may be conjugated and/or linked to any number from 1 to 64 (i.e., all) of the terminal groups of a G4 PAMAM. In certain embodiments, the PAMAM dendrimers have from 1% to 100% of their terminal groups conjugated and/or linked to metformin (e.g., via a linker (e.g., PEG)). In certain embodiments, the PAMAM dendrimers have from 35% to 100% of their terminal groups or surface functional groups conjugated and/or linked to metformin (e.g., via a linker (e.g., PEG)). In certain embodiments, the PAMAM dendrimers have from 1% to 35% of their terminal groups or surface functional groups conjugated and/or linked to metformin (e.g., via a linker (e.g., PEG)). In certain embodiments, the PAMAM dendrimers have at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 99%, or about 100% of their terminal groups or surface functional groups conjugated and/or linked to metformin (e.g., via a linker (e.g., PEG)).

In certain embodiments, the nanoparticle of the instant invention comprises a PAMAM dendrimer (e.g., a G4 PAMAM dendrimer) comprising amino terminal groups which are linked and/or conjugated to a linker (e.g., PEG) which is linked and/or conjugated to metformin (e.g., PAMAM-PEG-MET). In certain embodiments, the nanoparticle of the instant invention comprises the formula:

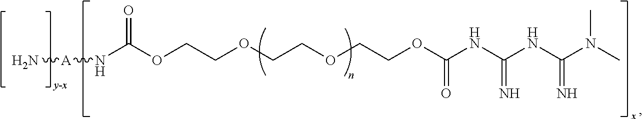

wherein A is a PAMAM dendrimer (e.g., a G4 PAMAM dendrimer), y is the number of terminal groups or surface functional groups of the PAMAM dendrimer, x is from 1 to y, and n is from 1-10. In certain embodiments, the nanoparticle of the instant invention comprises the formula:

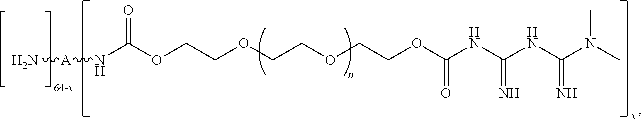

wherein A is a G4 PAMAM dendrimer, x is from 1 to 64, and n is from 1-10.

In certain embodiments, the nanoparticle of the instant invention comprises a PAMAM dendrimer (e.g., a G4 PAMAM dendrimer) comprising hydroxyl (e.g., amidoethanol) terminal groups linked and/or conjugated (e.g., via 1,1′-carbonyldiimidazole) to metformin (e.g., P4-MET). In certain embodiments, the nanoparticle of the instant invention comprises the formula:

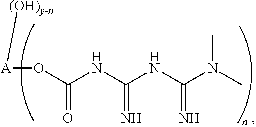

wherein A is a PAMAM dendrimer (e.g., a G4 PAMAM dendrimer), y is the number of terminal groups or surface functional groups of the PAMAM dendrimer, and n is from 1 to y. In certain embodiments, the nanoparticle of the instant invention comprises the formula:

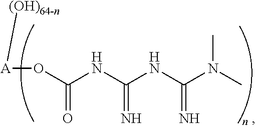

wherein A is a G4 PAMAM dendrimer and n is from 1 to 64.

The instant invention encompasses compositions (e.g., pharmaceutical compositions) comprising at least one nanoparticle of the instant invention and at least one carrier (e.g., pharmaceutically acceptable carrier). In certain embodiments, the composition comprises at least one nanoparticle of the instant invention and at least one pharmaceutically acceptable carrier. The compositions (e.g., pharmaceutical compositions) of the instant invention may further comprise other compounds or therapeutic agents.

As explained herein, the nanoparticles of the instant invention comprising metformin, such as PAMAM-PEG-MET and P4-MET, provide rapid distribution of the drug into tissue, and increased cellular uptake and penetration across blood-brain barrier than free metformin. These nanoparticles enable significantly increased drug delivery to irradiated tissues such as brain and significantly increased the overall survival rate of animals with glioblastoma compared to free metformin. The metformin nanoparticles of the instant invention can be used in medicine. More specifically, the metformin nanoparticles can be used to improve the efficacy of additional cancer therapies such as radiation therapy, chemotherapy, immunotherapy and targeted therapy. These metformin nanoparticles of the instant invention can also be used to drastically improve the modulation of macrophages activity compared with free metformin.

The present invention also encompasses methods for preventing, inhibiting, and/or treating a disease or disorder. The methods comprise administering a nanoparticle of the instant invention (e.g., in a composition) to a subject in need thereof. The diseases and disorders include, without limitation: cancer, immunological diseases and/or disorders, neurodegenerative disorders, and diabetes.

In certain embodiments, the methods prevent, inhibit, and/or treat cancer in a subject in need thereof. Cancers to be treated include, without limitation, brain cancer (e.g., gliomas, glioblastomas), CNS cancer, breast cancer, pancreatic cancer, colon cancer, ovarian cancer, prostate cancer, leukemias, lymphomas, lung cancer, liver cancer, stomach cancer, and skin cancer. In certain embodiments, the cancer is CNS cancer. In certain embodiments, the cancer is brain cancer. In certain embodiments, the cancer is a glioma. In certain embodiments, the cancer is a glioblastoma. In certain embodiments, the cancer is pancreatic cancer. In certain embodiments, the method comprises administering a nanoparticle of the instant invention (e.g., in a composition) to a subject in need thereof. In certain embodiments, the method further comprises administering at least one other therapeutic agent (e.g., anti-cancer agent, chemotherapeutic agent). In certain embodiments, the method further comprises administering at least one additional cancer therapy to the subject. Cancer therapies include, without limitation: radiation therapy, chemotherapy, immunotherapy and targeted therapy. When an additional therapy is administered, the additional therapy can be administered sequentially (e.g., before and/or after) and/or concurrently (e.g., simultaneously) with the administration of the nanoparticle or composition comprising the nanoparticle. In certain embodiments, the additional therapy is administered at least after the administration of the nanoparticle or composition comprising the nanoparticle (e.g., at least after the first dose of a regimen of the nanoparticle or composition comprising the nanoparticle). In certain embodiments, the additional therapy is administered at least during the same time frame as the administration of the nanoparticle or composition comprising the nanoparticle (e.g., at least during the regimen of the nanoparticle or composition comprising the nanoparticle).

In certain embodiments, the cancer treatment method comprises administering a nanoparticle of the instant invention (e.g., in a composition) and radiation therapy. In certain embodiments, the radiation therapy is CNS radiation therapy and the cancer is a CNS cancer. In certain embodiments, the radiation therapy is brain radiation therapy and the cancer is a brain cancer (e.g., gliomas, glioblastomas). In certain embodiments, the radiation therapy is administered at least after the administration of the nanoparticle or composition comprising the nanoparticle (e.g., at least after the first dose of a regimen of the nanoparticle or composition comprising the nanoparticle). In certain embodiments, the radiation therapy is administered at least during the same time frame as the administration of the nanoparticle or composition comprising the nanoparticle (e.g., at least during the regimen of the nanoparticle or composition comprising the nanoparticle).

In certain embodiments, the methods prevent, inhibit, and/or treat an immunological disease and/or disorder in a subject in need thereof. Immunological diseases and/or disorders include, without limitation: Crohn's, ulcerative colitis, rheumatoid arthritis, osteoarthritis, psoriasis, radiation pneumonitis, radiation necrosis, spinal cord injuries, and traumatic brain injuries. In certain embodiments, the disease is a neurodegenerative disorder. Neurodegenerative disorders include, without limitation: Alzheimer's disease, Parkinson's disease, Huntington's disease, Lewy body dementia and Amyotrophic Lateral Sclerosis. In certain embodiments, the method further comprises administering at least one additional therapy to the subject.

The nanoparticles of the instant invention (e.g., in a composition) can be administered to an animal, in particular a mammal, more particularly a human, in order to treat/inhibit/prevent the disease or disorder. The pharmaceutical compositions of the instant invention may also comprise at least one other compound or therapeutic agent. The additional compound may also be administered in a separate pharmaceutical composition from the compositions of the instant invention. The pharmaceutical compositions may be administered at the same time or at different times (e.g., sequentially).

The dosage ranges for the administration of the nanoparticles and/or compositions of the invention are those large enough to produce the desired effect (e.g., curing, relieving, treating, and/or preventing the disease or disorder, the symptoms of it, or the predisposition towards it). The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanoparticles described herein will generally be administered to a patient as a pharmaceutical composition. The term “patient” as used herein refers to human or animal subjects. These nanoparticles may be employed therapeutically, under the guidance of a physician.

The pharmaceutical compositions comprising the nanoparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the nanoparticles in the chosen medium may be varied and the medium may be chosen based on the desired route of administration of the pharmaceutical composition. Except insofar as any conventional media or agent is incompatible with the nanoparticles to be administered, its use in the pharmaceutical composition is contemplated.

The dose and dosage regimen of nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical composition will also depend upon the mode of administration chosen. For example, the nanoparticles of the invention may be administered by direct injection or to the bloodstream (e.g., intravenously). In this instance, a pharmaceutical composition comprises the nanoparticle dispersed in a medium that is compatible with the site of injection.

Nanoparticles of the instant invention may be administered by any method. For example, the nanoparticles of the instant invention can be administered, without limitation parenterally, subcutaneously, orally, topically, pulmonarily, rectally, vaginally, intravenously, intraperitoneally, intrathecally, intracerebrally, epidurally, intramuscularly, intradermally, or intracarotidly. In a particular embodiment, the nanoparticle is administered parenterally. In a particular embodiment, the nanoparticle is administered orally, intramuscularly, subcutaneously, or to the bloodstream (e.g., intravenously). In a particular embodiment, the nanoparticle is administered intramuscularly or subcutaneously. Pharmaceutical compositions for injection are known in the art. If injection is selected as a method for administering the nanoparticle, steps should be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a nanoparticle of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. In a particular embodiment, the nanoparticles of the instant invention, due to their long-acting therapeutic effect, need only be administered once to a subject.

Dosage units may be proportionately increased or decreased based on the weight of the patient. Appropriate concentrations for alleviation of a particular pathological condition may be determined by dosage concentration curve calculations, as known in the art.

The appropriate dosage unit for the administration of nanoparticles may be determined by evaluating their toxicity in animal models. Various concentrations of nanoparticles in pharmaceutical composition may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical composition comprising the nanoparticles may be administered at appropriate intervals until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., cancer) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of cancer herein may refer to curing, relieving, and/or preventing the cancer, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

“Linker” refers to a chemical moiety comprising a covalent bond or a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 0 (i.e., a bond) to about 100 atoms, from 0 to about 50 atoms, from 0 to about 10 atoms, or from about 1 to about 5 atoms.

The term “crosslinker” refers to a molecule capable of forming a covalent linkage between compounds.

The following examples provide illustrative methods of practicing the instant invention and are not intended to limit the scope of the invention in any way.

### Example 1

Metformin (1,1-dimethylbiguanide) is a synthetic biguanide and is an FDA-approved medicine for diabetes mellitus type 2. Metformin is recognized as a safe drug with the most prominent side effects being gastrointestinal-related side effects (e.g., abdominal pain, bloating, diarrhea, nausea, and/or vomiting) that occur in about 20-30% of patients.

The mitochondrion is the primary target of metformin (Vial, et al., Front. Endocrinol. (2019) 10:294). Notably, metformin can have anti-neoplastic properties at least partly through this mitochondrial action. Indeed, metformin exhibits a strong and consistent antiproliferative action on many types of cancer, including breast, colon, ovarian, pancreatic, lung and prostate cancer either in cell lines or in retrospective clinical studies. For example, metformin selectively affects glioblastoma tumor-initiating cells (cancer stem cells) and exerts antiproliferative activity on glioblastoma cells (Wurth et al., Cell Cycle (2013) 12(1):145-56). Additionally, metformin reduces the risk of cardiovascular disease (Lamanna et al., Diabetes Obes. Metab. (2011) 13(3):221-8), provides anti-aging properties, and can be therapeutic for central nervous system (CNS) diseases.

Despite these positive findings, metformin has failed to show significant clinical benefits in many prospectively conducted clinical trials. Significantly, the typical effective concentration of metformin in cell cultures is in the mM range, whereas the pharmacological concentrations in patients given oral metformin is typically less than 50 μM (Badrick, et al., Eur. J. Cancer (2014) 50(12):2119-25).

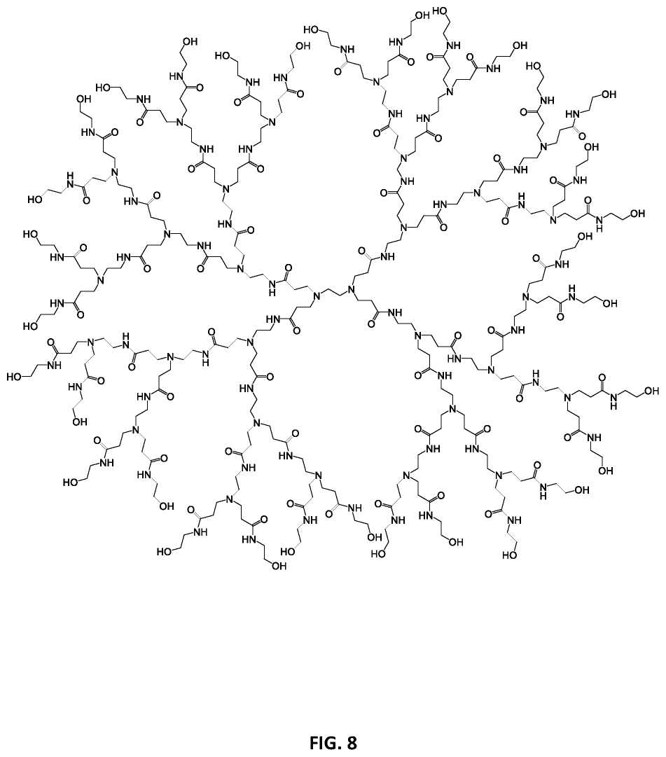

To improve delivery of metformin, polyamidoamine (PAMAM) dendrimers were employed. PAMAM dendrimers are hyperbranched polymers with molecular uniformity and narrow molecular weight distribution. The dendrimer PAMAM-OH G4 (Mw=14277.19 g/mole) was utilized. FIG. 8 provides the structure of PAMAM-OH G4.

In order to conjugate metformin to PAMAM-OH G4, conjugation via the 64 terminal —OH groups were exploited. Briefly, PAMAM-OH G4 (P4-OH) was first reacted with 1,1′-carbonyldiimidazole (CDI) and then reacted with metformin to yield PAMAM G4 conjugated metformin (P4-MET). A schematic of P4-MET showing the linkage of metformin is provided:

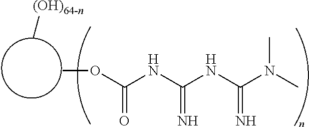

wherein the circle represents PAMAM G4 and n is from 1-64.

Dynamic light scattering (DLS) was used to determine the size of the dendrimers. P4-OH had a particle size (D_{eff}) of 7 nm±1 nm with a polydispersity index (PDI) of 0.17. P4-MET was determined to have a particle size (D_{eff}) of 13 nm±2 nm with a PDI of0.19.

Additionally, the zeta potential of the dendrimers was determined. P4-OH was determined to have a zeta potential of 0 mV and P4-MET was determined to have a zeta potential of 10 mV±2 mV.

Using an LH20 column, PAMAM-OH or the P4-MET conjugate were passed through the column and eluted with methanol. Appropriate fractions were collected, and the solvent was removed. PAMAM-OH and P4-MET were dried in vacuum and then analyzed and confirmed by ¹H-NMR (D₂O).

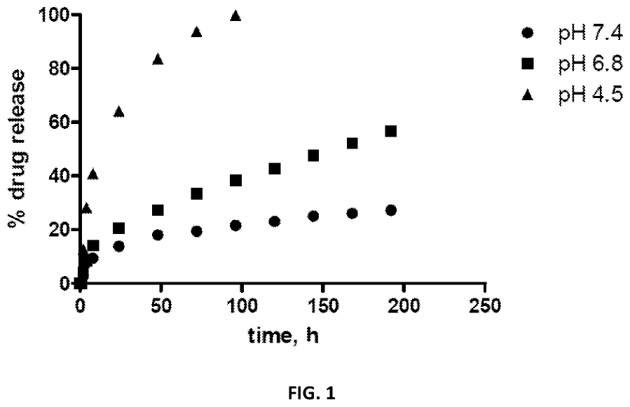

The ability of P4-MET to release metformin was then determined. As seen in FIG. 1, metformin was more rapidly released from P4-MET at acidic pH. Indeed, P4-MET retained over 50% of the conjugated metformin at pH 7.4 and pH 6.8 for over 150 hours. However, metformin was rapidly released from P4-MET at pH 4.5. The presence of lower pH in tumors and tumor environments can cause the release of more drug at the targeted site.

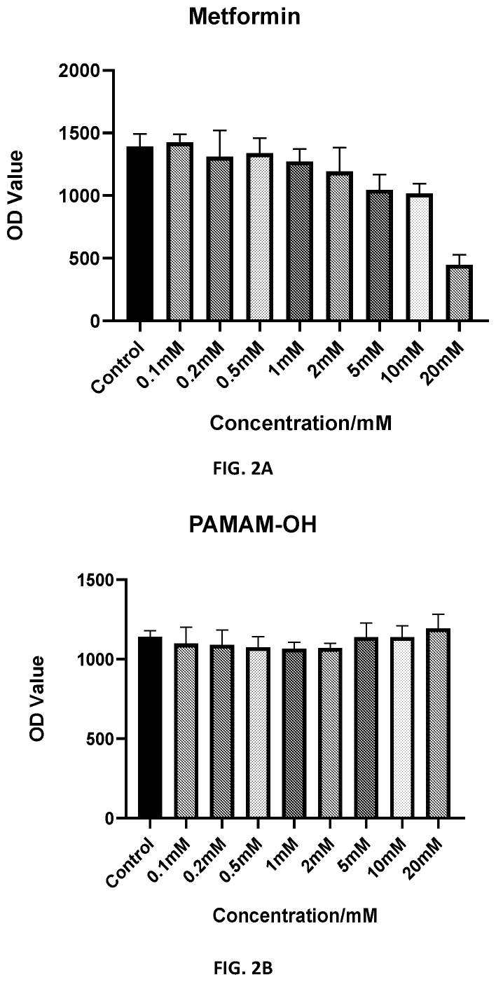

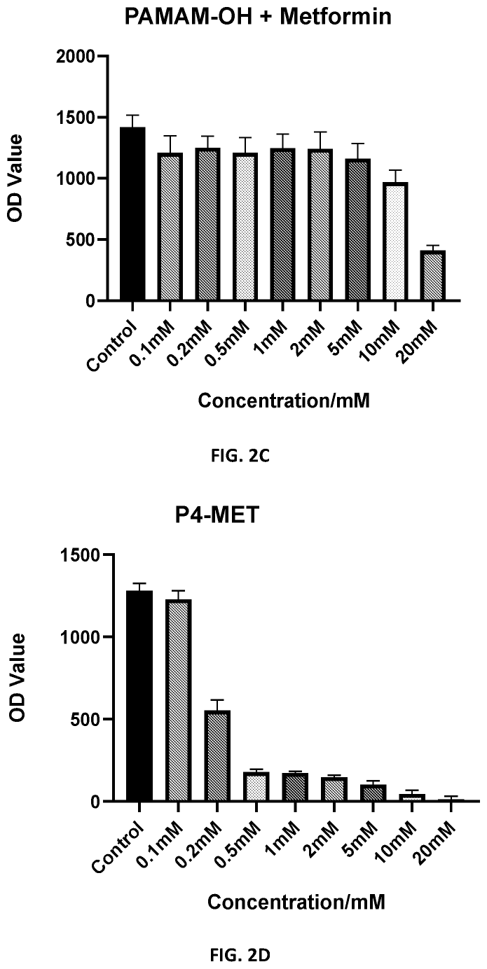

The ability of the dendrimers was tested for their ability to deliver metformin to U87MG cells, a human glioma cell line sensitive to metformin. Specifically, U87MG cells were exposed to metformin, PAMAM-OH, PAMAM-OH with metformin, or P4-MET. As seen in FIGS. 2A-2D, P4-MET greatly increased the delivery of metformin to U87MG cells as evidenced by the decreased optical density observed for the culture (e.g., less cell growth and/or more cell death). The IC₅₀ values are presented in Table 1.

TABLE 1
IC₅₀ values in U87MG cells.

| U87MG | IC₅₀ (mM) |
|---|---|
| metformin | 14.43 |
| PAMAM-OH | — |
| PAMAM-OH + metformin | 13.74 |
| P4-MET | 0.19 |

As seen in Table 1, P4-MET dramatically decreased (˜70×) the concentration of metformin needed to inhibit U87MG growth.

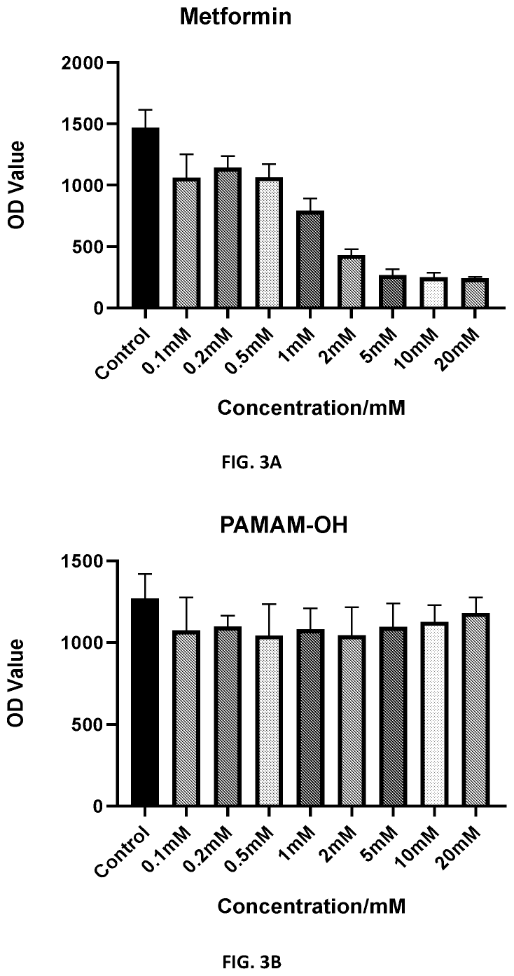

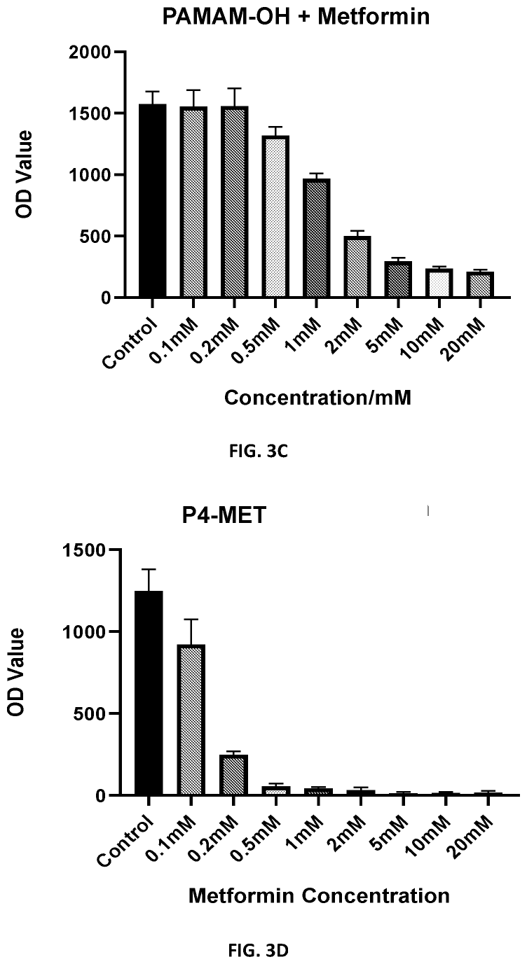

The ability of the dendrimers was also tested for their ability to deliver metformin to Ln229 cells, a human glioblastoma cell line sensitive to metformin. Specifically, Ln229 cells were exposed to metformin, PAMAM-OH, PAMAM-OH with metformin, or P4-MET. As seen in FIGS. 3A-3D, P4-MET greatly increased the delivery of metformin to LN229 cells as evidenced by the decreased optical density observed for the culture. The IC₅₀ values are presented in Table 2.

TABLE 2
IC₅₀ values in Ln229 cells.

| Ln229 | IC₅₀ (mM) |
|---|---|
| metformin | 0.99 |
| PAMAM-OH | — |
| PAMAM-OH + metformin | 1.46 |
| P4-MET | 0.13 |

As seen in Table 2, P4-MET dramatically decreased (˜7×) the concentration of metformin needed to inhibit Ln229 growth.

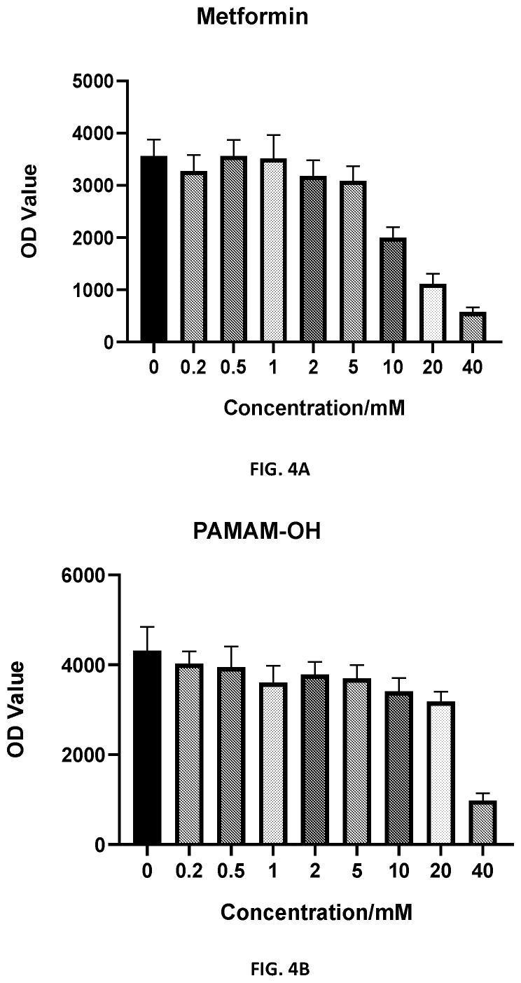

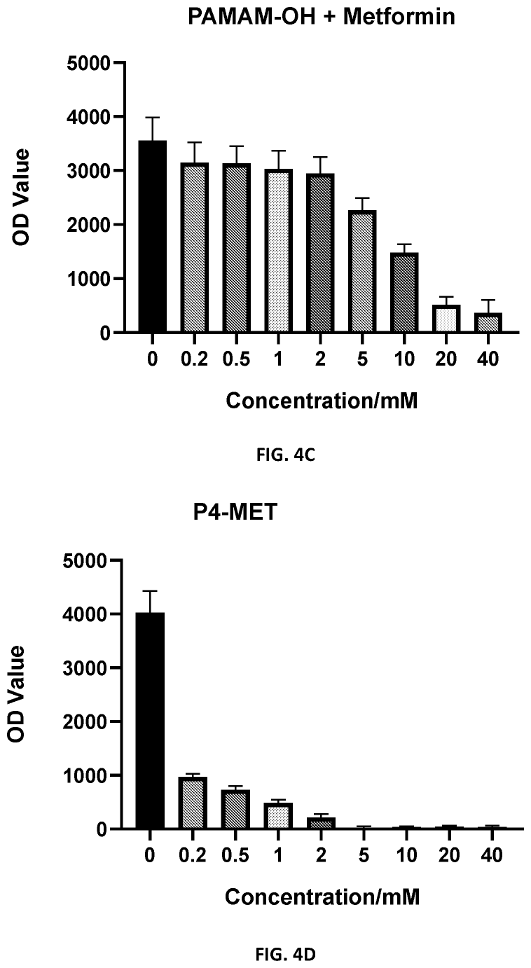

The ability of the dendrimers was also tested for their ability to deliver metformin to MGPP3 cells, a mouse glioblastoma cell line sensitive to metformin. Specifically, MGPP3 cells were exposed to metformin, PAMAM-OH, PAMAM-OH with metformin, or P4-MET. As seen in FIGS. 4A-4D, P4-MET greatly increased the delivery of metformin to MGPP3 cells as evidenced by the decreased optical density observed for the culture. The IC₅₀ values are presented in Table 3.

TABLE 3
IC₅₀ values in MGPP3 cells.

| MGPP3 | IC₅₀ (mM) |
|---|---|
| metformin | 12.52 |
| PAMAM-OH | 25.57 |
| PAMAM-OH + metformin | 6.75 |
| P4-MET | 0.05 |

As seen in Table 3, P4-MET dramatically decreased (˜250×) the concentration of metformin needed to inhibit MGPP3 growth.

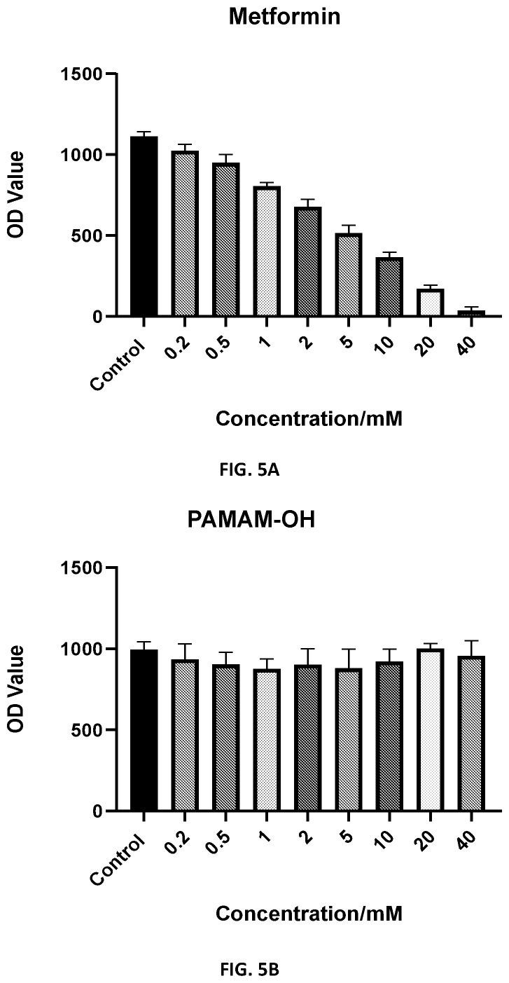

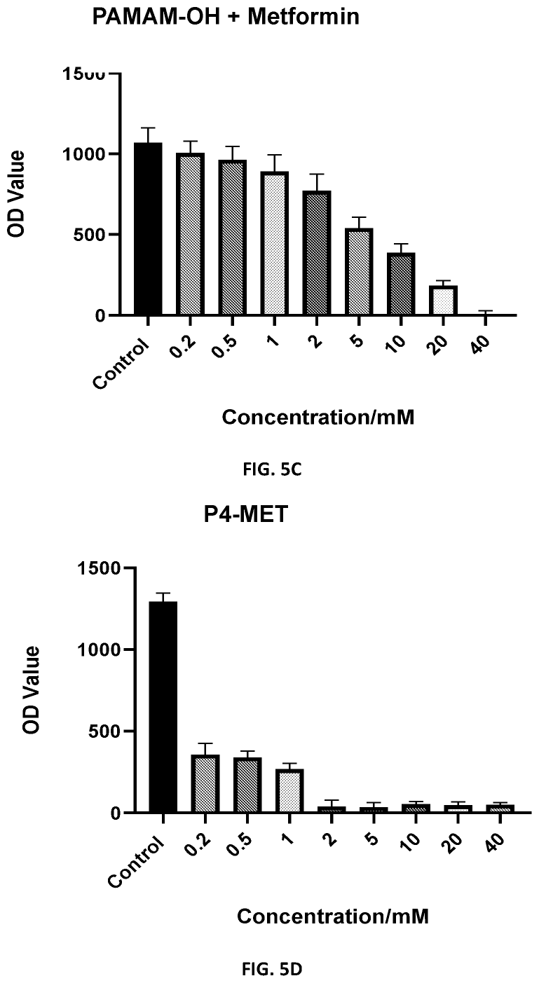

The ability of the dendrimers was also tested for their ability to deliver metformin to bone marrow-derived macrophages (BMDM). Specifically, BMDM cells were exposed to metformin, PAMAM-OH, PAMAM-OH with metformin, or P4-MET for 72 hours. As seen in FIGS. 5A-5D, P4-MET greatly increased the delivery of metformin to BMDM as evidenced by the decreased optical density observed for the culture. The IC₅₀ values are presented in Table 4.

TABLE 4
IC₅₀ values in BMDM cells.

| BMDM | IC₅₀ (mM) |
|---|---|
| metformin | 3.53 |
| PAMAM-OH | — |
| PAMAM-OH + metformin | 4.89 |
| P4-MET | 0.061 |

As seen in Table 4, P4-MET dramatically decreased (˜60×) the concentration of metformin needed to inhibit BMDM growth.

In addition, in vitro cellular uptake of P4-MET was confirmed by confocal microscopy. BMDM were exposed to Alexa Fluor® 488 P4-MET for 24 hours. Nuclei were stained with 4′,6-diamidino-2-phenylindole (DAPI). Mitochondria were stained with MitoTracker™ Deep Red. Alexa Fluor® 488 P4-MET staining and MitoTracker™ Deep Red staining overlapped.

The ability of the dendrimers was also tested for their ability to deliver metformin to UNKPC cells, a pancreatic cancer cell line. Specifically, MGPP3 cells were exposed to metformin, PAMAM-OH, or P4-MET. P4-MET greatly increased the delivery of metformin to UNKPC as evidenced by the decreased optical density observed for the culture. The IC₅₀ values are presented in Table 5.

TABLE 5
IC₅₀ values in UNKPC cells.

| UNKPC | IC₅₀ (mM) |
|---|---|
| metformin | 1.638 |
| PAMAM-OH | — |
| P4-MET | 0.1702 |

As seen in Table 5, P4-MET greatly decreased (˜10×) the concentration of metformin needed to inhibit UNKPC growth.

The ability of the dendrimers was also tested for their ability to deliver metformin to Panc-1 cells, a human pancreatic cancer cell line. Specifically, Panc-1 cells were exposed to metformin, PAMAM-OH, and P4-MET. P4-MET greatly increased the delivery of metformin to Panc-1 as evidenced by the decreased optical density observed for the culture. The IC₅₀ values are presented in Table 6.

TABLE 6
IC₅₀ values in UNKPC cells.

| Panc-1 | IC₅₀ (mM) |
|---|---|
| metformin | 5.244 |
| PAMAM-OH | — |
| P4-MET | 0.4024 |

As seen in Table 6, P4-MET greatly decreased (˜13×) the concentration of metformin needed to inhibit Panc-1 growth.

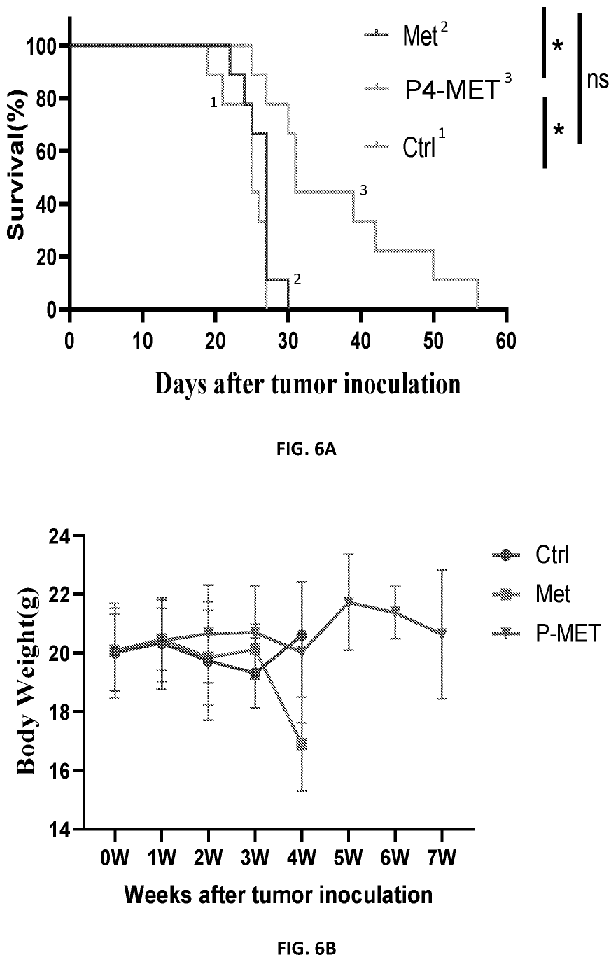

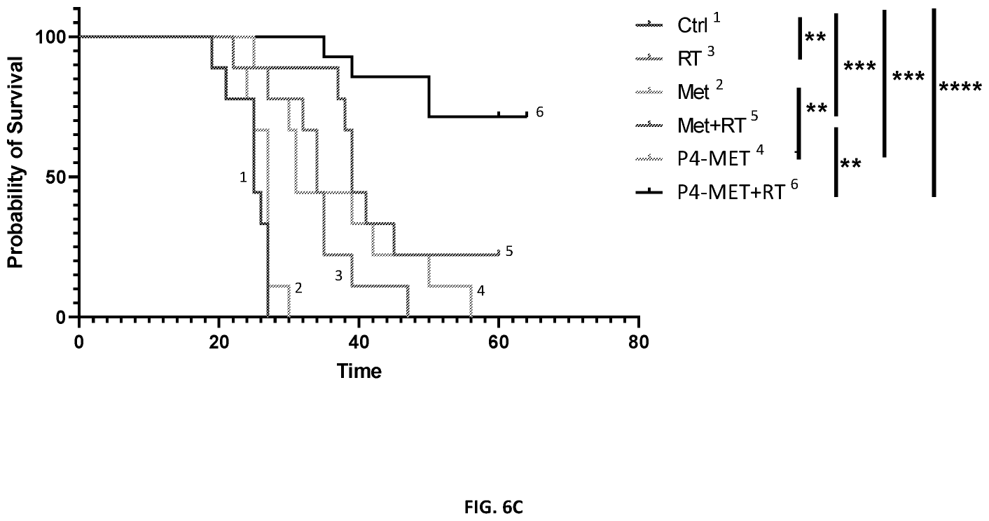

The ability of P4-MET to treat glioblastoma in vivo was also tested. Glioblastoma were implanted in mice at Day 0. At Day 8, mice were intraperitoneally administered vehicle (control) or 200 mg/kg metformin equivalents of metformin or P4-MET. Mice were treated daily until Day 28. As seen in FIG. 6A, mice treated with P4-MET had a statistically significant greater survival rate than mice treated with metformin. As seen in FIG. 6B, mice treated with P4-MET maintained their body weight through seven weeks of observation.

The mice were also treated with brain radiation therapy (RT). At Day 11 after tumor inoculation, the mice were treated with brain RT (8 Gy). As seen in FIG. 6C, P4-MET sensitizes brain RT and significantly improves overall survival in mice.

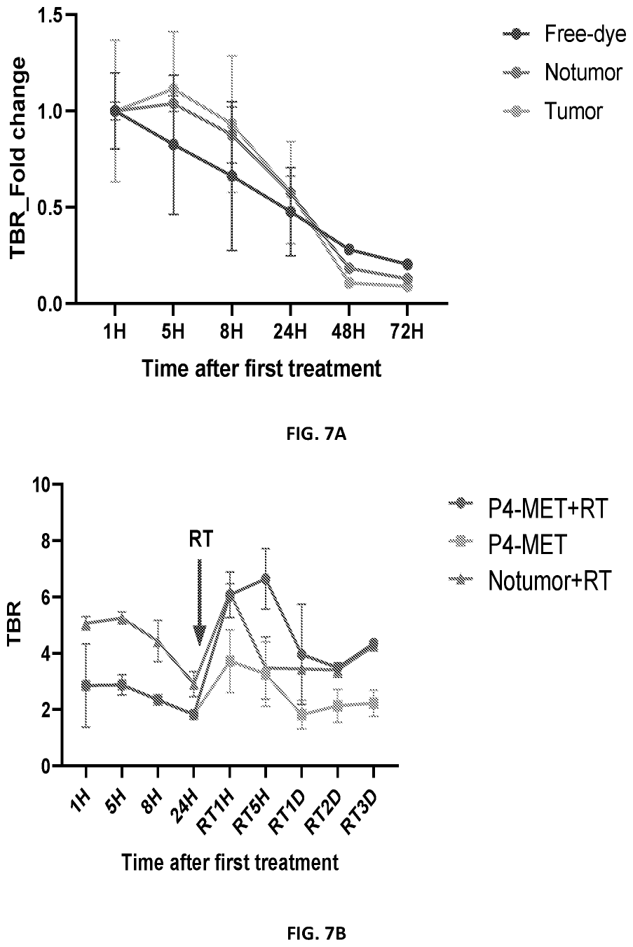

It was also determined whether brain radiation therapy increases P4-MET penetration. P4-MET was labeled with IRDye® (IR800) for visualization. FIG. 7A provides the tissue (brain)/background ratio (TBR) of P4-MET-IR800 after one injection. FIG. 7B provides the tissue (brain)/background ratio (TBR) of P4-MET-IR800 after a first injection at 0 hour and a second injection at 24 hours along with brain radiation therapy. As seen in FIG. 7B, brain RT increases the presence of P4-MET in the brain.

Lastly, it was determined that P4-MET was well tolerated by mice. Mice treated with P4-MET did not exhibit statistically significant differences in body weight, in blood cell counts, or a comprehensive metabolic panel (CMP) analysis.

In summary, P4-MET releases free metformin in acidic conditions. P4-MET can penetrate into cancer and benign cells (e.g., BMDM). While BMDM were very sensitive to P4-MET, significant adverse effects of P4-MET were not observed in mice. P4-MET significantly improved drug effects compared with free metformin at equivalent concentrations. Moreover, the increased P4-MET biological effects is significantly more than the additive effects of PAMAM and metformin when mixed together.

### Example 2

Another PAMAM conjugated to metformin was synthesized wherein metformin was attached to the PAMAM dendrimer via a polyethylene glycol (PEG) linker. Typically, a short PEG linker is used (e.g., wherein n=1-10). Briefly, PEG was first reacted with 1,1′-carbonyldiimidazole (CDI) to generate CDI-PEG-CDI.

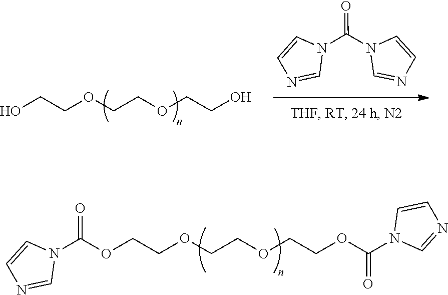

PAMAM G4 terminated with —NH₂ groups was then reacted with CDI-PEG-CDI to attach PEG-CDI to PAMAM-NH₂ via a carbamate bond.

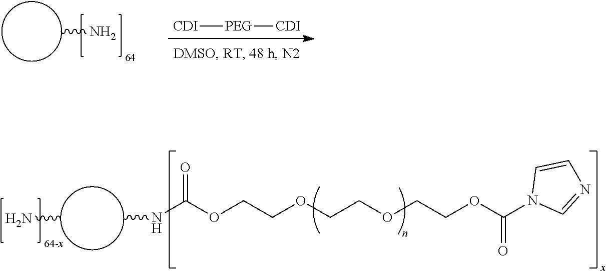

The circle represents PAMAM G4 and x is from 1-64. The product was then reacted with metformin (DMSO, RT, 48 h, N2) to yield PAMAM-PEG-metformin.

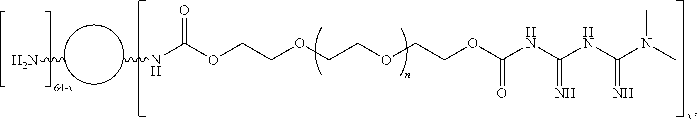

The circle represents PAMAM G4 and x is from 1-64. Products were analyzed and confirmed by ¹H-NMR.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A nanoparticle comprising a poly(amidoamine) (PAMAM) dendrimer conjugated to metformin.

2: The nanoparticle of claim 1, wherein said PAMAM dendrimer is a generation 3 PAMAM dendrimer, a generation 4 PAMAM dendrimer, or a generation 5 dendrimer.

3: The nanoparticle of claim 1, wherein said PAMAM dendrimer is a generation 4 PAMAM dendrimer.

4: The nanoparticle of claim 1, wherein said PAMAM dendrimer is conjugated to said metformin via a linker.

5: The nanoparticle of claim 4, wherein said linker comprises polyethylene glycol.

6: The nanoparticle of claim 4, wherein said linker comprises a carbamate bond.

7: The nanoparticle of claim 1, wherein the nanoparticle comprises the formula: 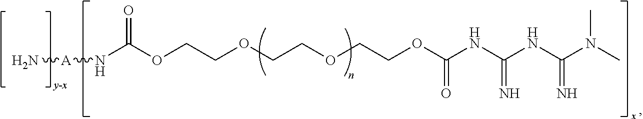
wherein A is a PAMAM dendrimer, y is the number of terminal groups of the PAMAM dendrimer, x is from 1 to y, and n is from 1 to 10.

8: The nanoparticle of claim 1, wherein the nanoparticle comprises the formula: 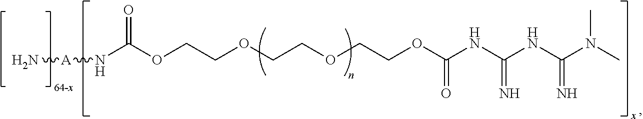
wherein A is a generation 4 PAMAM dendrimer, x is from 1 to 64, and n is from 1 to 10.

9: The nanoparticle of claim 1, wherein the nanoparticle comprises the formula: 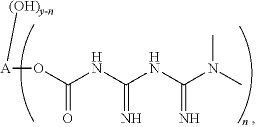
wherein A is a PAMAM dendrimer, y is the number of terminal groups of the PAMAM dendrimer, and n is from 1 to y.

10: The nanoparticle of claim 1, wherein the nanoparticle comprises the formula: 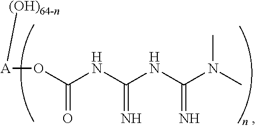
wherein A is a generation 4 PAMAM dendrimer and n is from 1 to 64.

11: A composition comprising at least nanoparticle of claim 1 and a pharmaceutically acceptable carrier.

12: A method of treating, inhibiting, and/or preventing a disease in a subject in need thereof, said method comprising administering to said subject a nanoparticle of claim 1.

13: The method of claim 12, wherein said disease is cancer.

14: The method of claim 13, wherein said cancer is a brain cancer.

15: The method of claim 13, wherein said cancer is a glioblastoma or glioma.

16: The method of claim 13, further comprising administering at least one of radiation therapy, chemotherapy, immunotherapy and targeted therapy.

17: The method of claim 13, further comprising administering radiation therapy.

18: The method of claim 13, further comprising administering brain radiation therapy, wherein said cancer is a brain cancer.

19: The method of claim 18, wherein said brain cancer is a glioblastoma or glioma.

20: The method of claim 12, wherein said disease is an immunological disorder.

21: The method of claim 20, wherein said immunological disorder is autoimmune disease.

22: The methods of claim 20, wherein said immunological disorder is rheumatoid arthritis, Crohn's disease, ulcerative colitis, psoriasis, multiple sclerosis, radiation pneumonitis, or radiation necrosis.

23: The method of claim 12, wherein said disease is a neurodegenerative disorder.

24: The method of claim 23, wherein said neurodegenerative disorder is dementia, radiation-, chemotherapy- or traumatic-brain-injury-induced neurocognitive deficits.
